(19) United States
(12) Patent Application Publication
(10) Pub. No.: US 2024/0156841 A1
(43) Pub. Date: May 16, 2024

(54) COATED NUT AND SEED PRODUCTS

(71) Applicant: AlmondRX LLC, San Diego, CA (US)

(72) Inventors: Frederick Allen Richburg, II, San Diego, CA (US); Amanda Richburg, San Diego, CA (US)

(21) Appl. No.: 18/510,237

(22) Filed: Nov. 15, 2023

Related U.S. Application Data
(60) Provisional application No. 63/425,628, filed on Nov. 15, 2022.

(51) Int. Cl. A61K 31/593; A23L 25/00; A23L 33/155; A61K 9/00; A61K 47/46

(52) U.S. Cl. CPC A61K 31/593; A23L 25/25; A23L 33/155; A61K 9/0056; A61K 47/46

(57) ABSTRACT

Disclosed are nut and seed products comprising a coating, wherein the coating comprises a nutritional additive, as well as methods of preparing said coated nut and seed products.

### RELATED APPLICATIONS

The present application claims the benefit of U.S. Provisional Application No. 63/425,628, filed Nov. 15, 2022, which is incorporated herein by reference in its entirety.

### FIELD OF THE INVENTION

The present disclosure is related to coated nut and seed products. The disclosure provides nuts and seeds that have a coating, where the coating includes a nutritional additive and an oil. The disclosure further provides methods of preparing coated nut and seed products.

### BACKGROUND

A balanced, healthy diet is required to provide the energy and nutrients required for one to survive and stay healthy. Maintaining a healthy diet requires the daily consumption of macronutrients, such as protein, fats, and carbohydrates, as well as micronutrients, such as vitamins, minerals, and trace elements. Micronutrient deficiencies can lead to a variety of health problems and conditions. For instance, vitamin D deficiency can lead to a loss of bone density and/or osteoporosis in adults, and rickets in children. However, some micronutrients, such as vitamin D, folic acid, vitamin A, vitamin B6, vitamin B12, calcium, vitamin E, iron, and iodine, are not present in many different natural foods, which could lead to deficiencies in these micronutrients in one's diet.

Therefore, the present disclosure addresses the unmet need for additional food sources that allow for the inclusion of an adequate supply of nutrients, particularly micronutrients, in adult and child diets.

### SUMMARY

Provided in the present disclosure are nut or seed products that include a coating, where the coating includes a nutritional additive and an oil.

In some embodiments, the nut or seed is selected from almond, peanut, pistachio, walnut, pecan, macadamia nut, cashew, pumpkin seed, and sunflower seed. In some embodiments, the nut or seed is an almond.

In some embodiments, the nutritional additive is selected from the group consisting of a vitamin, a mineral, an antioxidant agent, an anti-inflammatory agent, and combinations thereof.

In some embodiments, the nutritional additive is a vitamin selected from the group consisting of vitamin A, vitamin C, vitamin D, vitamin E, vitamin K, vitamin B1 (thiamine), vitamin B2 (riboflavin), vitamin B3 (niacin), vitamin B6 (pyridoxine), vitamin B12 (cyanocobalamin), pantothenic acid (B5), biotin (B7), folate (folic acid or B9), and combinations thereof. In some embodiments, the vitamin is vitamin D.

In some embodiments, the nutritional additive is a mineral selected from the group consisting of calcium, phosphorus, potassium, sodium, iodine, iron, magnesium, selenium, sulfur, copper, fluoride, chloride, manganese, cobalt, and combinations thereof.

In some embodiments, the nutritional additive is an antioxidant agent selected from the group consisting of vitamin C, vitamin E, beta-carotene, selenium, manganese, glutathione, coenzyme Q10, lipoic acid, flavonoids, phenols, polyphenols, phytoestrogens, and combinations thereof.

In some embodiments, the nutritional additive is an anti-inflammatory agent selected from the group consisting of omega-3 fatty acid, curcumin, S-adenosylmethionine, zinc, green tea, frankincense, capsaicin, cat's claw, and combinations thereof.

In some embodiments, the nut or seed product includes about 500 to about 1500 international units, about 600 to about 1400 international units, about 700 to about 1300 international units, about 800 to about 1200 international units, or about 900 to about 1100 international units of the nutritional additive per ounce of nut or seed. In some embodiments, the nut or seed product includes about 1000 international units of the nutritional additive per ounce of nut or seed.

In some embodiments, the nut or seed is skinless and/or shelled.

In some embodiments, the nut or seed is blanched.

In some embodiments, the nut or seed is roasted.

In some embodiments, the nut or seed is an almond, and the almond is a sweet almond.

In some embodiments, the sweet almond is selected from the group consisting of a nonpareil almond, a supareil almond, a Sonora almond, a Winters almond, an Avalon almond, a Durango almond, an Aldrich almond, a Price almond, a Wood Colony almond, a Carmel almond, a Monterey almond, a Butte almond, a Padre almond, a Mission almond, and a Ruby almond. In some embodiments, the almond is a nonpareil almond. In some embodiments, the almond is a supareil almond.

In some embodiments, the almond size is 16/18, 18/20, 20/22, 22/24, 23/25, 25/27, 27/30, 30/32, 32/34 or 36/40. In some embodiments, the almond size is 23/25.

In some embodiments, the oil is selected from the group consisting of vegetable oil, olive oil, canola oil, grapeseed oil, grape seed oil extract, sunflower oil, safflower oil, and combinations thereof. In some embodiments, the oil is selected from grapeseed oil, olive oil, sunflower oil, or combinations thereof.

In some embodiments, the nut or seed product further includes a flavor additive selected from salt, black pepper, garlic powder, Mexican spice, everything bagel seasoning, dark chocolate coconut, dark chocolate, honey cinnamon, cinnamon, 5 pepper blend, and combinations thereof. In some embodiments, the nut or seed product further includes a flavor additive selected from salt, black pepper, garlic powder, and combinations thereof.

In some embodiments, the flavor additive is salt. In some embodiments, the salt is sea salt.

In some embodiments, the nut or seed product includes about 0.1 to about 15.0 wt %, about 2.0 wt % to about 12.0 wt %, about 4.0 wt % to about 10.0 wt %, about 6.0 wt % to about 9.0 wt %, or about 7.5 wt % to about 8.5 wt % of the flavor additive.

In some embodiments, the nut or seed product includes about 8.0 wt % of the flavor additive.

In some embodiments, the nut or seed product includes about 0.01 wt % to about 2.0 wt %, about 0.05 wt % to about 1.5 wt %, about 0.1 wt % to about 1.0 wt %, about 0.2 wt % to about 0.8 wt %, about 0.3 wt % to about 0.7 wt %, or about 0.4 wt % to about 0.6 wt % of the flavor additive.

In some embodiments, the nut or seed product includes about 0.5 wt % of the flavor additive.

In some embodiments, the coating includes about 800 to about 1250 international units of a nutritional additive per ounce of almond, where the nutritional additive is vitamin D.

In some embodiments, the coating includes about 900 to about 1100 international units of a nutritional additive per ounce of almond, where the nutritional additive is vitamin D.

In some embodiments, the coating includes about 1000 international units of a nutritional additive per ounce of almond, where the nutritional additive is vitamin D.

In some embodiments, the coating includes about 400 international units of a nutritional additive per ounce of almond, where the nutritional additive is vitamin D.

Also provided in the present disclosure are skinless, blanched, roasted almonds that include a coating, where the coating includes:
about 800 to about 1250 international units of a nutritional additive per ounce of almond, where the nutritional additive is vitamin D;
about 0.2 wt % to about 1.2 wt % sunflower oil; and
about 0.1 wt % to about 15.0 wt % of a flavor additive;
where the almond is size 23/25.

Also provided in the present disclosure are skinless, blanched, roasted almonds that include a coating, where the coating includes:
about 900 to about 1100 international units of a nutritional additive per ounce of almond, where the nutritional additive is vitamin D;
about 0.2 wt % to about 1.2 wt % sunflower oil; and
about 0.1 wt % to about 1.0 wt % of a flavor additive, where the flavor additive is sea salt;
where the almond is size 23/25.

Also provided in the present disclosure are skinless, blanched, roasted almonds that include a coating, where the coating includes:
about 300 to about 500 international units of a nutritional additive per ounce of almond, where the nutritional additive is vitamin D;
about 0.2 wt % to about 1.2 wt % sunflower oil; and
about 0.1 wt % to about 1.0 wt % of a flavor additive, where the flavor additive is sea salt;
where the almond is size 23/25.

Also provided in the present disclosure are methods of preparing a nut or seed product, including:
a blanching step;
a roasting step; and
a seasoning step, including adding a coating including a nutritional additive to the nut or seed product.

In some embodiments of the method, the nut or seed product is almond.

In some embodiments of the method, the nutritional additive is added in oil form.

In some embodiments of the method, the nutritional additive is added in powdered form.

In some embodiments of the method, the nutritional additive is vitamin D.

In some embodiments of the method, the method includes adding about 800 to about 1250 international units of a nutritional additive per ounce of nut or seed product.

In some embodiments of the method, the method includes adding about 900 to about 1100 international units of a nutritional additive per ounce of nut or seed product.

In some embodiments of the method, the method includes adding about 300 to about 500 international units of a nutritional additive per ounce of nut or seed product.

In some embodiments of the method, the seasoning step further includes adding a flavor additive to the nut or seed product. In some embodiments of the method, the flavor additive is sea salt.

In some embodiments of the method, the method includes adding about 0.1 wt % to about 15.0 wt % of a flavor additive. In some embodiments of the method, the seasoning step further includes adding a tumbling oil.

In some embodiments of the method, the method includes adding about 0.09 wt % to about 0.2 wt % of a flavor additive. In some embodiments of the method, the seasoning step further includes adding a tumbling oil.

### DESCRIPTION OF DRAWINGS

FIG. 1 shows a flow-chart of an exemplary process for preparing coated almond products.

### DETAILED DESCRIPTION

Nuts and edible seeds are considered to be a healthy addition to any diet. Each variety of nut and seed is rich in plant-based protein and fiber, but also contains a unique combination of vitamins and minerals. As one example, almonds are among the most popularly consumed nut products. According to the United States Department of Agriculture (USDA), one ounce of almonds provides 164 calories, 6 grams protein, 14 grams fat (80% monounsaturated, 15% polyunsaturated, and 5% saturated), 6 grams carbohydrate, and 3 grams fiber, as well as 0.3 mg sodium, 60 mg calcium, 7 mg vitamin E, and 77 mg magnesium (https://fdc.nal.usda.gov/fdc-app.html#/food-details/323294/nutrients). Due to their high nutritional value, relative low cost, and ease of consumption, almonds are a popular health food around the world and may be an attractive option as a fortified food. The present disclosure relates to coated almond products, which include a coating, where the coating includes a nutritional additive. In some embodiments, the nutritional additive is a micronutrient. In some embodiments, the nutritional additive is a vitamin, a mineral, an antioxidant agent, an anti-inflammatory agent, or a combination thereof. In some embodiments, the almonds further include an oil and/or a flavor additive. The present disclosure further discloses methods of making the almonds. The almonds and methods of the present disclosure can be used to supplement one's diet with one or more micronutrients, for example, if there is a deficiency of micronutrients in one's diet. In some embodiments, the micronutrient is vitamin D.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### Coated Nut and Seed Products

Provided in the present disclosure are coated nut and seed products. The nut and seed products of the present disclosure have a coating that contains one or more nutritional additives and an oil. Thus, in some aspects, the disclosure provides a nut or seed product containing a coating, where the coating includes a nutritional additive and an oil.

The coated nut and seed product can include any type of edible nut or seed. Examples of edible nuts and seeds include, but are not limited to, almonds, peanuts, pistachios, walnuts, pecans, macadamia nuts, cashews, pumpkin seeds, and sunflower seeds.

In some embodiments, the coated nut and seed product is a coated nut product. In some embodiments, the coated nut product includes a nut selected from almond, peanut, pistachio, walnut, pecan, macadamia nut, and cashew. In some embodiments, the coated nut product is a coated almond product. In some embodiments, the coated nut product is a coated peanut product. In some embodiments, the coated nut product is a coated pistachio product. In some embodiments, the coated nut product is a coated walnut product. In some embodiments, the coated nut product is a coated pecan product. In some embodiments, the coated nut product is a coated macadamia nut product. In some embodiments, the coated nut product is a coated cashew product.

In some embodiments, the coated nut and seed product is a coated seed product. In some embodiments, the coated seed product includes a seed selected from sunflower seed and pumpkin seed. In some embodiments, the coated seed product is a coated sunflower seed product. In some embodiments, the coated seed product is a coated pumpkin seed product. The nutritional additive can be any nutritional additive that is not found naturally in a nut or seed, or any additive that is found naturally in a nut or seed that is to be supplemented. For example, the nutritional additive can be a nutrient or a micronutrient. In some embodiments, the nutritional additive is selected from the group consisting of a vitamin, a mineral, an antioxidant agent, an anti-inflammatory agent, and combinations thereof. In some embodiments, the nutritional additive is a vitamin. In some embodiments, the nutritional additive is a mineral. In some embodiments, the nutritional additive is an antioxidant agent. In some embodiments, the nutritional additive is an anti-inflammatory agent.

In some embodiments, the nutritional additive is a vitamin. In some embodiments, the vitamin is selected from the group consisting of vitamin A, vitamin C, vitamin D, vitamin E, vitamin K, vitamin B1 (thiamine), vitamin B2 (riboflavin), vitamin B3 (niacin), vitamin B6 (pyridoxine), vitamin B12 (cyanocobalamin), pantothenic acid (B5), biotin (B7), folate (folic acid or B9), and combinations thereof. In some embodiments, the nutritional additive is vitamin A. In some embodiments, the vitamin is vitamin C. In some embodiments, the vitamin is vitamin D. In some embodiments, the vitamin is vitamin E. In some embodiments, the vitamin is vitamin K. In some embodiments, the vitamin is vitamin B1 (thiamine). In some embodiments, the vitamin is vitamin B2 (riboflavin). In some embodiments, the vitamin is vitamin B3 (niacin). In some embodiments, the vitamin is vitamin B6 (pyridoxine). In some embodiments, the vitamin is vitamin B12 (cyanocobalamin). In some embodiments, the vitamin is pantothenic acid (B5). In some embodiments, the vitamin is biotin (B7). In some embodiments, the vitamin is folate (folic acid or B9).

In some embodiments, the nutritional additive is vitamin D.

In some embodiments, vitamin D is vitamin D2. In some embodiments, vitamin D is vitamin D3. In some embodiments, vitamin D includes vitamin D2 and vitamin D3.

In some embodiments, the nutritional additive is a mineral. In some embodiments, the mineral is selected from the group consisting of calcium, phosphorus, potassium, sodium, iodine, iron, magnesium, selenium, sulfur, copper, fluoride, chloride, manganese, cobalt, and combinations thereof. In some embodiments, the mineral is calcium. In some embodiments, the mineral is phosphorus. In some embodiments, the mineral is potassium. In some embodiments, the mineral is sodium. In some embodiments, the mineral is iodine. In some embodiments, the mineral is iron. In some embodiments, the mineral is magnesium. In some embodiments, the mineral is selenium. In some embodiments, the mineral is sulfur. In some embodiments, the mineral is copper. In some embodiments, the mineral is fluoride. In some embodiments, the mineral is chloride. In some embodiments, the mineral is manganese. In some embodiments, the mineral is cobalt.

In some embodiments, the nutritional additive is an antioxidant agent. In some embodiments, the antioxidant agent is selected from the group consisting of vitamin C, vitamin E, beta-carotene, selenium, manganese, glutathione, coenzyme Q10, lipoic acid, a flavonoid, a phenol, a polyphenol, a phytoestrogen, and combinations thereof. In some embodiments, the antioxidant agent is vitamin C. In some embodiments, the antioxidant agent is vitamin E. In some embodiments, the antioxidant agent is beta-carotene. In some embodiments, the antioxidant agent is selenium. In some embodiments, the antioxidant agent is manganese. In some embodiments, the antioxidant agent is glutathione. In some embodiments, the antioxidant agent is coenzyme Q10. In some embodiments, the antioxidant agent is lipoic acid. In some embodiments, the antioxidant agent is a flavonoid. In some embodiments, the antioxidant agent is a phenol. In some embodiments, the antioxidant agent is a polyphenol. In some embodiments, the antioxidant agent is a phytoestrogen.

In some embodiments, the flavonoid is selected from the group consisting of flavanols, flavan-3-ols, flavones, flavanones, isoflavanones, anthocyanins, and combinations thereof.

In some embodiments, the phenol is selected from the group consisting of tannins, chalcones, coumarins, phenolic acids, catechins, anthocyanins, proanthocyanins, and combinations thereof.

In some embodiments, the polyphenol is selected from the group consisting of quercetin, catechins, capsaicinoids, stilbenes, and combinations thereof.

In some embodiments, the phytoestrogen is selected from the group consisting of genistein, daidzein, glycitein, formononetin, biochanin, lignans, and combinations thereof.

In some embodiments, the nutritional additive is an anti-inflammatory agent.

In some embodiments, the anti-inflammatory agent is selected from the group consisting of omega-3 fatty acid, curcumin, S-adenosylmethionine, zinc, green tea, frankincense, capsaicin, cat's claw, and combinations thereof. In some embodiments, the anti-inflammatory agent is omega-3 fatty acid. In some embodiments, the anti-inflammatory agent is curcumin. In some embodiments, the anti-inflammatory agent is S-adenosylmethionine. In some embodiments, the anti-inflammatory agent is zinc. In some embodiments, the anti-inflammatory agent is green tea. In some embodiments, the anti-inflammatory agent is frankincense. In some embodiments, the anti-inflammatory agent is capsaicin. In some embodiments, the anti-inflammatory agent is cat's claw.

In some embodiments, the coating contains one or more nutritional additives, such as one, two, three, four, five or more nutritional additives. In some embodiments, the coating includes one nutritional additive. In some embodiments, the coating contains more than one nutritional additive. In some embodiments, the coating includes two nutritional additives. In some embodiments, the coating includes three nutritional additives. In some embodiments, the coating includes four nutritional additives. In some embodiments, the coating includes five nutritional additives.

In some embodiments, the coating includes one more nutritional additives, where at least one nutritional additive is a vitamin. In some embodiments, the coating includes two nutritional additives, where at least one nutritional additive is a vitamin. In some embodiments, the coating includes three nutritional additives, where at least one nutritional additive is a vitamin. In some embodiments, the coating includes four nutritional additives, where at least one nutritional additive is a vitamin. In some embodiments, the coating includes five nutritional additives, where at least one nutritional additive is a vitamin.

In some embodiments, the coating includes two nutritional additives, where at least one nutritional additive is vitamin D. In some embodiments, the coating includes three nutritional additives, where at least one nutritional additive is vitamin D. In some embodiments, the coating includes one more nutritional additives, where at least one nutritional additive is vitamin D. In some embodiments, the coating includes four nutritional additives, where at least one nutritional additive is vitamin D. In some embodiments, the coating includes five nutritional additives, where at least one nutritional additive is vitamin D.

In some embodiments, the coating includes about 500 to about 1500 international units, about 600 to about 1400 international units, about 700 to about 1300 international units, about 800 to about 1200 international units, or about 900 to about 1100 international units of the nutritional additive per ounce of nut or seed. In some embodiments, the coating includes about 500 to about 1500 international units of the nutritional additive per ounce of nut or seed. In some embodiments, the coating includes about 600 to about 1400 international units of the nutritional additive per ounce of nut or seed. In some embodiments, the coating includes about 700 to about 1300 international units of the nutritional additive per ounce of nut or seed. In some embodiments, the coating includes about 800 to about 1250 international units of the nutritional additive per ounce of nut or seed. In some embodiments, the coating includes about 800 to about 1200 international units of the nutritional additive per ounce of nut or seed. In some embodiments, the coating includes about 800 to about 1250 international units of the nutritional additive per ounce of nut or seed. In some embodiments, the coating includes about 900 to about 1100 international units of the nutritional additive per ounce of nut or seed. In some embodiments, the nut or seed is an almond.

In some embodiments, the coating includes about 500 international units of the nutritional additive per ounce of nut or seed. In some embodiments, the coating includes about 600 international units of the nutritional additive per ounce of nut or seed. In some embodiments, the coating includes about 700 international units of the nutritional additive per ounce of nut or seed. In some embodiments, the coating includes about 800 international units of the nutritional additive per ounce of nut or seed. In some embodiments, the coating includes about 900 international units of the nutritional additive per ounce of nut or seed. In some embodiments, the coating includes about 1000 international units of the nutritional additive per ounce of nut or seed. In some embodiments, the coating includes about 1100 international units of the nutritional additive per ounce of nut or seed. In some embodiments, the coating includes about 1200 international units of the nutritional additive per ounce of nut or seed. In some embodiments, the coating includes about 1250 international units of the nutritional additive per ounce of nut or seed. In some embodiments, the coating includes about 1300 international units of the nutritional additive per ounce of nut or seed. In some embodiments, the coating includes about 1400 international units of the nutritional additive per ounce of nut or seed. In some embodiments, the coating includes about 1500 international units of the nutritional additive per ounce of nut or seed. In some embodiments, the nut or seed is an almond.

In some embodiments, the coating includes about 50 to about 750 international units, about 100 to about 700 international units, about 150 to about 650 international units, about 200 to about 600 international units, about 250 to about 550 international units, about 300 to about 500 international units, or about 350 to about 450 international units of the nutritional additive per ounce of nut or seed.

In some embodiments, the coating includes about 50 international units of the nutritional additive per ounce of nut or seed. In some embodiments, the coating includes 100 international units of the nutritional additive per ounce of nut or seed. In some embodiments, the coating includes 150 international units of the nutritional additive per ounce of nut or seed. In some embodiments, the coating includes 200 international units of the nutritional additive per ounce of nut or seed. In some embodiments, the coating includes 250 international units of the nutritional additive per ounce of nut or seed. In some embodiments, the coating includes 300 international units of the nutritional additive per ounce of nut or seed. In some embodiments, the coating includes 350 international units of the nutritional additive per ounce of nut or seed. In some embodiments, the coating includes 400 international units of the nutritional additive per ounce of nut or seed. In some embodiments, the coating includes 450 international units of the nutritional additive per ounce of nut or seed. In some embodiments, the coating includes 500 international units of the nutritional additive per ounce of nut or seed. In some embodiments, the coating includes 550 international units of the nutritional additive per ounce of nut or seed. In some embodiments, the coating includes 600 international units of the nutritional additive per ounce of nut or seed. In some embodiments, the coating includes 650 international units of the nutritional additive per ounce of nut or seed. In some embodiments, the coating includes 700 international units of the nutritional additive per ounce of nut or seed. In some embodiments, the coating includes 750 international units of the nutritional additive per ounce of nut or seed.

In some embodiments, the coating includes about 0.001 wt % to about 0.1 wt %, about 0.005 wt % to about 0.07 wt %, about 0.007 wt % to about 0.05 wt %, or about 0.009 wt % to about 0.03 wt % of the nutritional additive per ounce of nut or seed. In some embodiments, the coating includes about 0.001 wt % to about 0.1 wt %. In some embodiments, the coating includes about 0.005 wt % to about 0.07 wt % of the nutritional additive per ounce of nut or seed. In some embodiments, the coating includes about 0.007 wt % to about 0.05 wt % of the nutritional additive per ounce of nut or seed. In some embodiments, the coating includes about 0.009 wt % to about 0.03 wt % of the nutritional additive per ounce of nut or seed. In some embodiments, the nut or seed is an almond.

In some embodiments, the coating includes about 0.001 wt %. In some embodiments, the coating includes about 0.005 wt % of the nutritional additive per ounce of nut or seed. In some embodiments, the coating includes about 0.007 wt % of the nutritional additive per ounce of nut or seed. In some embodiments, the coating includes about 0.009 wt % of the nutritional additive per ounce of nut or seed. In some embodiments, the coating includes about 0.03 wt % of the nutritional additive per ounce of nut or seed. In some embodiments, the coating includes about 0.05 wt % of the nutritional additive per ounce of nut or seed. In some embodiments, the coating includes about 0.07 wt % of the nutritional additive per ounce of nut or seed. In some embodiments, the coating includes about 0.1 wt % of the nutritional additive per ounce of nut or seed. In some embodiments, the nut or seed is an almond.

In some embodiments, the coating includes about 500 to about 1500 international units, about 600 to about 1400 international units, about 700 to about 1300 international units, about 800 to about 1200 international units, about 800 to about 1250 international units, or about 900 to about 1100 international units of vitamin D per ounce of nut or seed. In some embodiments, the coating includes about 200 to about 1800 international units of vitamin D per ounce of nut or seed. In some embodiments, the coating includes about 500 to about 1500 international units of vitamin D per ounce of nut or seed. In some embodiments, the coating includes about 600 to about 1400 international units of vitamin D per ounce of nut or seed. In some embodiments, the coating includes about 700 to about 1300 international units of vitamin D per ounce of nut or seed. In some embodiments, the coating includes about 800 to about 1200 international units of vitamin D per ounce of nut or seed. In some embodiments, the coating includes about 800 to about 1250 international units of vitamin D per ounce of nut or seed. In some embodiments, the coating includes about 900 to about 1100 international units of vitamin D per ounce of nut or seed. In some embodiments, the nut or seed is an almond.

In some embodiments, the coating includes about 500 international units of vitamin D per ounce of nut or seed. In some embodiments, the coating includes about 600 international units of vitamin D per ounce of nut or seed. In some embodiments, the coating includes about 700 international units of vitamin D per ounce of nut or seed. In some embodiments, the coating includes about 800 international units of vitamin D per ounce of nut or seed. In some embodiments, the coating includes about 900 international units of vitamin D per ounce of nut or seed. In some embodiments, the coating includes about 1000 international units of vitamin D per ounce of nut or seed. In some embodiments, the coating includes about 1100 international units of vitamin D per ounce of nut or seed. In some embodiments, the coating includes about 1200 international units of vitamin D per ounce of nut or seed. In some embodiments, the coating includes about 1250 international units of vitamin D per ounce of nut or seed. In some embodiments, the coating includes about 1300 international units of vitamin D per ounce of nut or seed. In some embodiments, the coating includes about 1400 international units of vitamin D per ounce of nut or seed. In some embodiments, the coating includes about 1500 international units of vitamin D per ounce of nut or seed. In some embodiments, the nut or seed is an almond.

In some embodiments, the coating includes about 50 to about 750 international units of vitamin D, about 100 to about 700 international units of vitamin D, about 150 to about 650 international units of vitamin D, about 200 to about 600 international units of vitamin D, about 250 to about 550 international units of vitamin D, about 300 to about 500 international units of vitamin D, or about 350 to about 450 international units of vitamin D per ounce of nut or seed. In some embodiments, the nut or seed is an almond.

In some embodiments, the coating includes about 50 international units of vitamin D per ounce of nut or seed. In some embodiments, the coating includes 100 international units of vitamin D per ounce of nut or seed. In some embodiments, the coating includes 150 international units of vitamin D per ounce of nut or seed. In some embodiments, the coating includes 200 international units of vitamin D per ounce of nut or seed. In some embodiments, the coating includes 250 international units of vitamin D per ounce of nut or seed. In some embodiments, the coating includes 300 international units of vitamin D per ounce of nut or seed. In some embodiments, the coating includes 350 international units of vitamin D per ounce of nut or seed. In some embodiments, the coating includes 400 international units of vitamin D per ounce of nut or seed. In some embodiments, the coating includes 450 international units of vitamin D per ounce of nut or seed. In some embodiments, the coating includes 500 international units of vitamin D per ounce of nut or seed. In some embodiments, the coating includes 550 international units of vitamin D per ounce of nut or seed. In some embodiments, the coating includes 600 international units of vitamin D per ounce of nut or seed. In some embodiments, the coating includes 650 international units of vitamin D per ounce of nut or seed. In some embodiments, the coating includes 700 international units of vitamin D per ounce of nut or seed. In some embodiments, the coating includes 750 international units of vitamin D per ounce of nut or seed. In some embodiments, the nut or seed is an almond.

In some embodiments, the coating includes about 0.001 wt % to about 0.1 wt %, about 0.005 wt % to about 0.07 wt %, about 0.007 wt % to about 0.05 wt %, or about 0.009 wt % to about 0.03 wt % of vitamin D per ounce of nut or seed. In some embodiments, the coating includes about 0.001 wt % to about 0.1 wt %. In some embodiments, the coating includes about 0.005 wt % to about 0.07 wt % of vitamin D per ounce of nut or seed. In some embodiments, the coating includes about 0.007 wt % to about 0.05 wt % of vitamin D per ounce of nut or seed. In some embodiments, the coating includes about 0.009 wt % to about 0.03 wt % of vitamin D per ounce of nut or seed. In some embodiments, the nut or seed is an almond.

In some embodiments, the coating includes about 0.001 wt %. In some embodiments, the coating includes about 0.005 wt % of vitamin D per ounce of nut or seed. In some embodiments, the coating includes about 0.007 wt % of vitamin D per ounce of nut or seed. In some embodiments, the coating includes about 0.009 wt % of vitamin D per ounce of nut or seed. In some embodiments, the coating includes about 0.03 wt % of vitamin D per ounce of nut or seed. In some embodiments, the coating includes about 0.05 wt % of vitamin D per ounce of nut or seed. In some embodiments, the coating includes about 0.07 wt % of vitamin D per ounce of nut or seed. In some embodiments, the coating includes about 0.1 wt % of vitamin per ounce of nut or seed. In some embodiments, the nut or seed is an almond.

In some embodiments, the coating includes about 0.1 μg to about 1 g of nutritional additive per ounce of nut or seed. In some embodiments, the coating includes about 1μg to about 100 μg of nutritional additive per ounce of nut or seed. In some embodiments, the coating includes about 5 μg to about 95 μg of nutritional additive per ounce of nut or seed. In some embodiments, the coating includes about 10 μg to about 90 μg of nutritional additive per ounce of nut or seed. In some embodiments, the coating includes about 20 μg to about 80 μg of nutritional additive per ounce of nut or seed. In some embodiments, the coating includes about 40 μg to about 60 μg of nutritional additive per ounce of nut or seed. In some embodiments, the coating includes about 1 mg to about 1000 mg of nutritional additive per ounce of nut or seed. In some embodiments, the coating includes about 10 mg to about 900 mg of nutritional additive per ounce of nut or seed. In some embodiments, the coating includes about 50 mg to about 800 mg of nutritional additive per ounce of nut or seed. In some embodiments, the coating includes about 100 mg to about 700 mg of nutritional additive per ounce of nut or seed. In some embodiments, the coating includes about 200 mg to about 600 mg of nutritional additive per ounce of nut or seed. In some embodiments, the coating includes about 300 mg to about 500 mg of nutritional additive per ounce of nut or seed. In some embodiments, the nut or seed is an almond.

In some embodiments, the coating includes about 10 μg to about 40 μg of vitamin D per ounce of nut or seed. In some embodiments, the coating includes about 12.5 μg to about 37.5 μg of vitamin D per ounce of nut or seed. In some embodiments, the coating includes about 15 μg to about 35 μg of vitamin D per ounce of nut or seed. In some embodiments, the coating includes about 20 μg to about 30 μg of vitamin D per ounce of nut or seed. In some embodiments, the coating includes about 22.5 μg to about 27.5 μg of vitamin D per ounce of nut or seed. In some embodiments, the nut or seed is an almond.

In some embodiments, the coating includes about 10 μg of vitamin D per ounce of nut or seed. In some embodiments, the coating includes about 12.5 μg of vitamin D per ounce of nut or seed. In some embodiments, the coating includes about 15 μg of vitamin D per ounce of nut or seed. In some embodiments, the coating includes about 20 μg of vitamin D per ounce of nut or seed. In some embodiments, the coating includes about 22.5 μg of vitamin D per ounce of nut or seed. In some embodiments, the coating includes about 25 μg of vitamin D per ounce of nut or seed. In some embodiments, the coating includes about 27.5 μg of vitamin D per ounce of nut or seed. In some embodiments, the coating includes about 30 μg of vitamin D per ounce of nut or seed. In some embodiments, the coating includes about 35 μg of vitamin D per ounce of nut or seed. In some embodiments, the coating includes about 37.5 μg of vitamin D per ounce of nut or seed. In some embodiments, the coating includes about 40 μg of vitamin D per ounce of nut or seed. In some embodiments, the nut or seed is an almond.

In some embodiments, the oil is selected from the group consisting of vegetable oil, olive oil, canola oil, grapeseed oil, grape seed oil extract, sunflower oil, safflower oil, and combinations thereof. In some embodiments, the oil is vegetable oil. In some embodiments, the oil is olive oil. In some embodiments, the oil is canola oil. In some embodiments, the oil is grapeseed oil. In some embodiments, the oil is grape seed oil extract. In some embodiments, the oil is sunflower oil. In some embodiments, the oil is safflower oil.

In some embodiments, the coating includes grapeseed oil.

In some embodiments, the coating includes olive oil.

In some embodiments, the coating includes sunflower oil. In some embodiments, the coating includes olive oil and sunflower oil.

In some embodiments, the coating includes about 0.01 wt % to about 2.0 wt %, about 0.05 wt % to about 1.5 wt %, about 0.1 wt % to about 1.2 wt %, about 0.3 wt % to about 1.0 wt %, about 0.5 wt % to about 0.9 wt %, or about 0.6 wt % to about 0.8 wt % of the oil. In some embodiments, the coating includes about 0.01 wt % to about 2.0 wt %. In some embodiments, the coating includes about 0.05 wt % to about 1.5 wt % of the oil. In some embodiments, the coating includes about 0.1 wt % to about 1.2 wt % of the oil. In some embodiments, the coating includes about 0.3 wt % to about 1.0 wt % of the oil. In some embodiments, the coating includes about 0.5 wt % to about 0.9 wt % of the oil. In some embodiments, the coating includes about 0.6 wt % to about 0.8 wt % of the oil.

In some embodiments, the coating includes about 0.01 wt %. In some embodiments, the coating includes about 0.05 wt % of the oil. In some embodiments, the coating includes about 0.1 wt % of the oil. In some embodiments, the coating includes about 0.3 wt % of the oil. In some embodiments, the coating includes about 0.5 wt % of the oil. In some embodiments, the coating includes about 0.6 wt % of the oil. In some embodiments, the coating includes about 0.7 wt % of the oil. In some embodiments, the coating includes about 0.8 wt % of the oil. In some embodiments, the coating includes about 0.9 wt % of the oil. In some embodiments, the coating includes about 1.0 wt % of the oil. In some embodiments, the coating includes about 1.2 wt % of the oil. In some embodiments, the coating includes about 1.5 wt % of the oil. In some embodiments, the coating includes about 2.0 wt % of the oil.

In some embodiments, the coating includes about 0.01 wt % to about 2.0 wt %, about 0.05 wt % to about 1.5 wt %, about 0.1 wt % to about 1.2 wt %, about 0.3 wt % to about 1.0 wt %, about 0.5 wt % to about 0.9 wt %, or about 0.6 wt % to about 0.8 wt % of grapeseed oil. In some embodiments, the coating includes about 0.01 wt % to about 2.0 wt %. In some embodiments, the coating includes about 0.05 wt % to about 1.5 wt % of grapeseed oil. In some embodiments, the coating includes about 0.1 wt % to about 1.2 wt % of grapeseed oil. In some embodiments, the coating includes about 0.3 wt % to about 1.0 wt % of grapeseed oil. In some embodiments, the coating includes about 0.5 wt % to about 0.9 wt % of grapeseed oil. In some embodiments, the coating includes about 0.6 wt % to about 0.8 wt % of grapeseed oil.

In some embodiments, the coating includes about 0.01 wt %. In some embodiments, the coating includes about 0.05 wt % of grapeseed oil. In some embodiments, the coating includes about 0.1 wt % of grapeseed oil. In some embodiments, the coating includes about 0.3 wt % of grapeseed oil. In some embodiments, the coating includes about 0.5 wt % of grapeseed oil. In some embodiments, the coating includes about 0.6 wt % of grapeseed oil. In some embodiments, the coating includes about 0.7 wt % of grapeseed oil. In some embodiments, the coating includes about 0.8 wt % of grapeseed oil. In some embodiments, the coating includes about 0.9 wt % of grapeseed oil. In some embodiments, the coating includes about 1.0 wt % of grapeseed oil. In some embodiments, the coating includes about 1.2 wt % of grapeseed oil. In some embodiments, the coating includes about 1.5 wt % of grapeseed oil. In some embodiments, the coating includes about 2.0 wt % of grapeseed oil.

In some embodiments, the coating includes about 0.01 wt % to about 2.0 wt %, about 0.05 wt % to about 1.5 wt %, about 0.1 wt % to about 1.2 wt %, about 0.3 wt % to about 1.0 wt %, about 0.5 wt % to about 0.9 wt %, or about 0.6 wt % to about 0.8 wt % of olive oil. In some embodiments, the coating includes about 0.01 wt % to about 2.0 wt %. In some embodiments, the coating includes about 0.05 wt % to about 1.5 wt % of olive oil. In some embodiments, the coating includes about 0.1 wt % to about 1.2 wt % of olive oil. In some embodiments, the coating includes about 0.3 wt % to about 1.0 wt % of olive oil. In some embodiments, the coating includes about 0.5 wt % to about 0.9 wt % of olive oil. In some embodiments, the coating includes about 0.6 wt % to about 0.8 wt % of olive oil.

In some embodiments, the coating includes about 0.01 wt %. In some embodiments, the coating includes about 0.05 wt % of olive oil. In some embodiments, the coating includes about 0.1 wt % of olive oil. In some embodiments, the coating includes about 0.3 wt % of olive oil. In some embodiments, the coating includes about 0.5 wt % of olive oil. In some embodiments, the coating includes about 0.6 wt % of olive oil. In some embodiments, the coating includes about 0.7 wt % of olive oil. In some embodiments, the coating includes about 0.8 wt % of olive oil. In some embodiments, the coating includes about 0.9 wt % of olive oil. In some embodiments, the coating includes about 1.0 wt % of olive oil. In some embodiments, the coating includes about 1.2 wt % of olive oil. In some embodiments, the coating includes about 1.5 wt % of olive oil. In some embodiments, the coating includes about 2.0 wt % of olive oil.

In some embodiments, the coating includes about 0.01 wt % to about 2.0 wt %, about 0.05 wt % to about 1.5 wt %, about 0.1 wt % to about 1.2 wt %, about 0.3 wt % to about 1.0 wt %, about 0.5 wt % to about 0.9 wt %, or about 0.6 wt % to about 0.8 wt % of sunflower oil. In some embodiments, the coating includes about 0.01 wt % to about 2.0 wt %. In some embodiments, the coating includes about 0.05 wt % to about 1.5 wt % of sunflower oil. In some embodiments, the coating includes about 0.1 wt % to about 1.2 wt % of sunflower oil. In some embodiments, the coating includes about 0.3 wt % to about 1.0 wt % of sunflower oil. In some embodiments, the coating includes about 0.5 wt % to about 0.9 wt % of sunflower oil. In some embodiments, the coating includes about 0.6 wt % to about 0.8 wt % of sunflower oil.

In some embodiments, the coating includes about 0.01 wt %. In some embodiments, the coating includes about 0.05 wt % of sunflower oil. In some embodiments, the coating includes about 0.1 wt % of sunflower oil. In some embodiments, the coating includes about 0.3 wt % of sunflower oil. In some embodiments, the coating includes about 0.5 wt % of sunflower oil. In some embodiments, the coating includes about 0.6 wt % of sunflower oil. In some embodiments, the coating includes about 0.7 wt % of sunflower oil. In some embodiments, the coating includes about 0.8 wt % of sunflower oil. In some embodiments, the coating includes about 0.9 wt % of sunflower oil. In some embodiments, the coating includes about 1.0 wt % of sunflower oil. In some embodiments, the coating includes about 1.2 wt % of sunflower oil. In some embodiments, the coating includes about 1.5 wt % of sunflower oil. In some embodiments, the coating includes about 2.0 wt % of sunflower oil.

In some embodiments, the coating includes about 0.01 wt % to about 2.0 wt %, about 0.05 wt % to about 1.5 wt %, about 0.1 wt % to about 1.2 wt %, about 0.3 wt % to about 1.0 wt %, about 0.5 wt % to about 0.9 wt %, or about 0.6 wt % to about 0.8 wt % of olive oil and sunflower oil. In some embodiments, the coating includes about 0.01 wt % to about 2.0 wt %. In some embodiments, the coating includes about 0.05 wt % to about 1.5 wt % of olive oil and sunflower oil. In some embodiments, the coating includes about 0.1 wt % to about 1.2 wt % of olive oil and sunflower oil. In some embodiments, the coating includes about 0.3 wt % to about 1.0 wt % of olive oil and sunflower oil. In some embodiments, the coating includes about 0.5 wt % to about 0.9 wt % of olive oil and sunflower oil. In some embodiments, the coating includes about 0.6 wt % to about 0.8 wt % of olive oil and sunflower oil.

In some embodiments, the coating includes about 0.01 wt %. In some embodiments, the coating includes about 0.05 wt % of olive oil and sunflower oil. In some embodiments, the coating includes about 0.1 wt % of olive oil and sunflower oil. In some embodiments, the coating includes about 0.3 wt % of olive oil and sunflower oil. In some embodiments, the coating includes about 0.5 wt % of olive oil and sunflower oil. In some embodiments, the coating includes about 0.6 wt % of olive oil and sunflower oil. In some embodiments, the coating includes about 0.7 wt % of olive oil and sunflower oil. In some embodiments, the coating includes about 0.8 wt % of olive oil and sunflower oil. In some embodiments, the coating includes about 0.9 wt % of olive oil and sunflower oil. In some embodiments, the coating includes about 1.0 wt % of olive oil and sunflower oil. In some embodiments, the coating includes about 1.2 wt % of olive oil and sunflower oil. In some embodiments, the coating includes about 1.5 wt % of olive oil and sunflower oil. In some embodiments, the coating includes about 2.0 wt % of olive oil and sunflower oil.

In some embodiments, the coating includes a flavor additive.

In some embodiments, the nut or seed product further includes a flavor additive selected from salt, black pepper, garlic powder, Mexican spice, everything bagel seasoning, dark chocolate coconut, dark chocolate, honey cinnamon, cinnamon, 5 pepper blend, and combinations thereof.

In some embodiments, the flavor additive is selected from salt, black pepper, garlic powder, and combinations thereof. In some embodiments, the flavor additive is salt. In some embodiments, the flavor additive is black pepper. In some embodiments, the flavor additive is garlic powder.

In some embodiments, the Mexican spice may comprise cayenne pepper, cumin, coriander, chili powder, garlic powder, onion powder, red pepper flakes, paprika, achiote paste, salt, cinnamon, or cloves, and combinations thereof.

In some embodiments, the everything bagel seasoning may comprise poppy seeds, sesame seeds, black sesame seeds, minced dried garlic, minced dried onion, or salt, and combinations thereof.

In some embodiments, the 5 pepper blend may comprise Tellicherry, pink, green, white, and Jamaican peppercorns.

In some embodiments, the flavor additive is black pepper. In some embodiments, the flavor additive is garlic powder. In some embodiments, the flavor additive is Mexican spice. In some embodiments, the flavor additive is everything bagel. In some embodiments, the flavor additive is dark chocolate coconut. In some embodiments, the flavor additive is dark chocolate. In some embodiments, the flavor additive is honey cinnamon. In some embodiments, the flavor additive is cinnamon. In some embodiments, the flavor additive is 5 pepper blend.

In some embodiments, the coating further includes salt. In some embodiments, the coating further includes sea salt.

In some embodiments, the nut or seed product includes about 0.1 wt % to about 15.0 wt %, about 2.0 wt % to about 12.0 wt %, about 4.0 wt % to about 10.0 wt %, about 6.0 wt % to about 9.0 wt %, or about 7.5 wt % to about 8.5 wt % of the flavor additive.

In some embodiments, the nut or seed product includes about 0.1 wt % of the flavor additive. In some embodiments, the nut or seed product includes about 15.0 wt % of the flavor additive. In some embodiments, the nut or seed product includes about 2.0 wt % of the flavor additive. In some embodiments, the nut or seed product includes about 12.0 wt % of the flavor additive. In some embodiments, the nut or seed product includes about 4.0 wt % of the flavor additive. In some embodiments, the nut or seed product includes about 10.0 wt % of the flavor additive. In some embodiments, the nut or seed product includes about 6.0 wt % of the flavor additive. In some embodiments, the nut or seed product includes about 9.0 wt % of the flavor additive. In some embodiments, the nut or seed product includes about 7.5 wt % of the flavor additive. In some embodiments, the nut or seed product includes about 8.5 wt % of the flavor additive.

In some embodiments, the nut or seed product includes about 8.0 wt % of the flavor additive.

In some embodiments, the coating includes about 0.01 wt % to about 2.0 wt %, about 0.05 wt % to about 1.5 wt %, about 0.1 wt % to about 1.0 wt %, about 0.2 wt % to about 0.8 wt %, about 0.3 wt % to about 0.7 wt %, or about 0.4 wt % to about 0.6 wt % of the flavor additive. In some embodiments, the coating includes about 0.01 wt % to about 2.0 wt % of the flavor additive. In some embodiments, the coating includes about 0.5 wt % to about 1.5 wt % of the flavor additive. In some embodiments, the coating includes about 0.1 wt % to about 1.0 wt % of the flavor additive. In some embodiments, the coating includes about 0.2 wt % to about 0.8 wt % of the flavor additive. In some embodiments, the coating includes about 0.3 wt % to about 0.7 wt % of the flavor additive. In some embodiments, the coating includes about 0.4 wt % to about 0.6 wt % of the flavor additive.

In some embodiments, the coating includes about 0.01 wt % of the flavor additive. In some embodiments, the coating includes about 0.04 wt % of the flavor additive. In some embodiments, the coating includes about 0.06 wt % of the flavor additive. In some embodiments, the coating includes about 0.08 wt % of the flavor additive. In some embodiments, the coating includes about 0.09 wt % of the flavor additive. In some embodiments, the coating includes about 0.1 wt % of the flavor additive. In some embodiments, the coating includes about 0.2 wt % of the flavor additive. In some embodiments, the coating includes about 0.3 wt % of the flavor additive. In some embodiments, the coating includes about 0.5 wt % of the flavor additive. In some embodiments, the coating includes about 1.0 wt % of the flavor additive. In some embodiments, the coating includes about 1.5 wt % of the flavor additive. In some embodiments, the coating includes about 2.0 wt % of the flavor additive.

In some embodiments, the coating includes about 0.01 wt % to about 2.0 wt %, about 0.05 wt % to about 1.5 wt %, about 0.1 wt % to about 1.0 wt %, about 0.2 wt % to about 0.8 wt %, about 0.3 wt % to about 0.7 wt %, or about 0.4 wt % to about 0.6 wt % of salt. In some embodiments, the coating includes about 0.01 wt % to about 2.0 wt % of salt. In some embodiments, the coating includes about 0.05 wt % to about 1.5 wt % of salt. In some embodiments, the coating includes about 0.1 wt % to about 1.0 wt % of salt. In some embodiments, the coating includes about 0.2 wt % to about 0.8 wt % of salt. In some embodiments, the coating includes about 0.3 wt % to about 0.7 wt % of salt. In some embodiments, the coating includes about 0.4 wt % to about 0.6 wt % of salt.

In some embodiments, the coating includes about 0.01 wt % of salt. In some embodiments, the coating includes about 0.04 wt % of salt. In some embodiments, the coating includes about 0.06 wt % of salt. In some embodiments, the coating includes about 0.08 wt % of salt. In some embodiments, the coating includes about 0.09 wt % of salt. In some embodiments, the coating includes about 0.1 wt % of salt. In some embodiments, the coating includes about 0.2 wt % of salt. In some embodiments, the coating includes about 0.3 wt % of salt. In some embodiments, the coating includes about 0.5 wt % of salt. In some embodiments, the coating includes about 1.0 wt % of salt. In some embodiments, the coating includes about 1.5 wt % of salt. In some embodiments, the coating includes about 2.0 wt % of salt.

In some embodiments, the nutritional additive is vitamin D, and the coating further includes a flavor additive. In some embodiments, the nutritional additive is vitamin D, and the coating further includes salt. In some embodiments, the nutritional additive is vitamin D, and the coating further includes sea salt. In some embodiments, the nutritional additive is vitamin D, the oil is grapeseed oil, and the coating further includes a flavor additive. In some embodiments, the nutritional additive is vitamin D, the oil is grapeseed oil, and the coating further includes salt. In some embodiments, the nutritional additive is vitamin D, the oil is grapeseed oil, and the coating further includes sea salt. In some embodiments, the coating further includes sea salt. In some embodiments, the oil is grapeseed oil and the coating further includes a flavor additive. In some embodiments, the oil is grapeseed oil and the coating further includes salt. In some embodiments, the oil is grapeseed oil and the coating further includes sea salt. In some embodiments, the nutritional additive is vitamin D, the oil is olive oil, and the coating further includes a flavor additive. In some embodiments, the nutritional additive is vitamin D, the oil is olive oil, and the coating further includes salt. In some embodiments, the nutritional additive is vitamin D, the oil is olive oil, and the coating further includes sea salt. In some embodiments, the oil is olive oil and the coating further includes a flavor additive. In some embodiments, the oil is olive oil and the coating further includes salt. In some embodiments, the oil is olive oil and the coating further includes sea salt. In some embodiments, the nutritional additive is vitamin D, the oil is sunflower oil, and the coating further includes a flavor additive. In some embodiments, the nutritional additive is vitamin D, the oil is sunflower oil, and the coating further includes salt. In some embodiments, the nutritional additive is vitamin D, the oil is sunflower oil, and the coating further includes sea salt. In some embodiments, the oil is sunflower oil and the coating further includes a flavor additive. In some embodiments, the oil is sunflower oil and the coating further includes salt. In some embodiments, the oil is sunflower oil and the coating further includes sea salt. In some embodiments, the nutritional additive is vitamin D, the oil is olive oil and sunflower oil, and the coating further includes a flavor additive. In some embodiments, the nutritional additive is vitamin D, the oil is olive oil and sunflower oil, and the coating further includes salt. In some embodiments, the nutritional additive is vitamin D, the oil is olive oil and sunflower oil, and the coating further includes sea salt. In some embodiments, the oil is olive oil and sunflower oil, and the coating further includes a flavor additive. In some embodiments, the oil is olive oil and sunflower oil, and the coating further includes salt. In some embodiments, the oil is olive oil and sunflower oil, and the coating further includes sea salt.

In some embodiments, the nut or seed product is skinless.

In some embodiments, the nut or seed product is shelled.

In some embodiments, the nut or seed product is blanched.

In some embodiments, the nut or seed product is roasted.

In some embodiments, the nut or seed product is shelled and blanched.

In some embodiments, the nut or seed product is skinless and blanched.

In some embodiments, the nut or seed product is shelled and roasted.

In some embodiments, the nut or seed product is skinless and roasted.

In some embodiments, the nut or seed product is blanched and roasted.

In some embodiments, the nut or seed product is shelled, blanched, and roasted.

In some embodiments, the nut or seed product is skinless, blanched, and roasted. In some embodiments, the nut or seed product is shelled, skinless, blanched, and roasted.

In some embodiments, the coated nut and seed product is a coated almond product, where any almond type or almond size is suitable for use in the embodiments of the present disclosure.

In some embodiments, the almond is skinless.

In some embodiments, the almond is blanched.

In some embodiments, the almond is roasted.

In some embodiments, the almond is skinless and blanched.

In some embodiments, the almond is skinless and roasted.

In some embodiments, the almond is blanched and roasted.

In some embodiments, the almond is skinless, blanched, and roasted.

In some embodiments, the almond is a sweet almond.

In some embodiments, the almond is selected from the group consisting of a nonpareil almond, a supareil almond, a Sonora almond, a Winters almond, an Avalon almond, a Durango almond, an Aldrich almond, a Price almond, a Wood Colony almond, a Carmel almond, a Monterey almond, a Butte almond, a Padre almond, a Mission almond, and a Ruby almond. In some embodiments, the almond is a nonpareil almond. In some embodiments, the almond is a supareil almond. In some embodiments, the almond is a Sonora almond. In some embodiments, the almond is a Winters almond. In some embodiments, the almond is an Avalon almond. In some embodiments, the almond is a Durango almond. In some embodiments, the almond is an Aldrich almond. In some embodiments, the almond is a Price almond. In some embodiments, the almond is a Wood Colony almond. In some embodiments, the almond is a Carmel almond. In some embodiments, the almond is a Monterey almond. In some embodiments, the almond is a Butte almond. In some embodiments, the almond is a Padre almond. In some embodiments, the almond is a Mission almond. In some embodiments, the almond is a Ruby almond.

In some embodiments, the almond size is 16/18, 18/20, 20/22, 22/24, 23/25, 25/27, 27/30, 30/32, 32/34 or 36/40. In some embodiments, the almond size is 16/18. In some embodiments, the almond size is 18/20. In some embodiments, the almond size is 20/22. In some embodiments, the almond size is 22/24. In some embodiments, the almond size is 23/25. In some embodiments, the almond size is 25/27. In some embodiments, the almond size is 27/30. In some embodiments, the almond size is 30/32. In some embodiments, the almond size is 32/34. In some embodiments, the almond size is 36/40.

In some embodiments, the coated nut or seed product is a coated almond product, and the almond is a skinless almond including a coating, where the coating includes about 800 to about 1250 international units of vitamin D.

In some embodiments, the coated nut or seed product is a coated almond product, and the almond is a skinless almond including a coating, where the coating includes about 900 to about 1100 international units of vitamin D.

In some embodiments, the coated nut or seed product is a coated almond product, and the almond is a skinless almond including a coating, where the coating includes about 1000 international units of vitamin D.

In some embodiments, the coated nut or seed product is a coated almond product, and the almond is a skinless, blanched, roasted almond including a coating, where the coating includes about 800 to about 1250 international units of vitamin D.

In some embodiments, the coated nut or seed product is a coated almond product, and the almond is a skinless, blanched, roasted almond including a coating, where the coating includes about 900 to about 1100 international units of vitamin D.

In some embodiments, the coated nut or seed product is a coated almond product, and the almond is a skinless, blanched, roasted almond including a coating, where the coating includes about 1000 international units of vitamin D.

In some embodiments, the coated nut or seed product is a coated almond product, and the almond is a skinless, blanched, roasted almond including a coating, where the coating includes about 800 to about 1250 international units of vitamin D and about 0.1 wt % to about 15.0 wt % of a flavor additive.

In some embodiments, the coated nut or seed product is a coated almond product, and the almond is a skinless, blanched, roasted almond including a coating, where the coating includes about 900 to about 1100 international units of vitamin D and about 0.1 wt % to about 15.0 wt % of a flavor additive.

In some embodiments, the coated nut or seed product is a coated almond product, and the almond is a skinless, blanched, roasted almond including a coating, where the coating includes about 1000 international units of vitamin D and about 0.5 wt % of salt.

In some embodiments, the coated nut or seed product is a coated almond product, and the almond is a skinless, blanched, roasted almond including a coating, where the coating includes about 800 to about 1250 international units of vitamin D and about 0.1 wt % to about 15.0 wt % of a flavor additive.

In some embodiments, the coated nut or seed product is a coated almond product, and the almond is a skinless, blanched, roasted almond including a coating, where the coating includes about 900 to about 1100 international units of vitamin D and about 0.1 wt % to about 1.0 wt % of salt.

In some embodiments, the coated nut or seed product is a coated almond product, and the almond is a skinless, blanched, roasted almond including a coating, where the coating includes about 1000 international units of vitamin D and about 0.5 wt % of salt.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of grapeseed oil;
where the almond is size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of grapeseed oil;
where the almond is size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of grapeseed oil;
where the almond is size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of grapeseed oil;
where the almond is a nonpareil almond and the almond size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of grapeseed oil;
where the almond is a nonpareil almond and the almond size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of grapeseed oil;
where the almond is a nonpareil almond and the almond size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of grapeseed oil;
where the almond is a supareil almond and the almond size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of grapeseed oil;
where the almond is a supareil almond and the almond size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of grapeseed oil;
where the almond is a supareil almond and the almond size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of olive oil;
where the almond is size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of olive oil;
where the almond is size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of olive oil;
where the almond is size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of olive oil;
where the almond is a nonpareil almond and the almond size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of olive oil;
where the almond is a nonpareil almond and the almond size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of olive oil;
where the almond is a nonpareil almond and the almond size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of olive oil;
where the almond is a supareil almond and the almond size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of olive oil;
where the almond is a supareil almond and the almond size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of olive oil;
where the almond is a supareil almond and the almond size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of sunflower oil;
where the almond is size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of sunflower oil;
where the almond is size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of sunflower oil;
where the almond is size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of sunflower oil;
where the almond is a nonpareil almond and the almond size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of sunflower oil;
where the almond is a nonpareil almond and the almond size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of sunflower oil;
where the almond is a nonpareil almond and the almond size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of sunflower oil;
where the almond is a supareil almond and the almond size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of sunflower oil;
where the almond is a supareil almond and the almond size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of sunflower oil;
where the almond is a supareil almond and the almond size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of olive oil and sunflower oil;
where the almond is size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of olive oil and sunflower oil;
where the almond is size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of olive oil and sunflower oil;
where the almond is size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of olive oil and sunflower oil;
where the almond is a nonpareil almond and the almond size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of olive oil and sunflower oil;
where the almond is a nonpareil almond and the almond size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of olive oil and sunflower oil;
where the almond is a nonpareil almond and the almond size 23/25.

In some embodiments, the coated nut or seed product is a coated almond product, and the almond is a skinless, blanched, roasted almond including a coating, where the coating includes:
about 800 to about 1250 international units of vitamin D;
about 0.1 wt % to about 1.0 wt % of salt; and
about 0.2 wt % to about 1.2 wt % of olive oil and sunflower oil;
where the almond is a supareil almond and the almond size 23/25.

In some embodiments, the coated nut or seed product is a coated almond product, and the almond is a skinless, blanched, roasted almond including a coating, where the coating includes:
about 900 to about 1100 international units of vitamin D;
about 0.1 wt % to about 1.0 wt % of salt; and
about 0.2 wt % to about 1.2 wt % of olive oil and sunflower oil;
where the almond is a supareil almond and the almond size 23/25.

In some embodiments, the coated nut or seed product is a coated almond product, and the almond is a skinless, blanched, roasted almond including a coating, where the coating includes:
about 1000 international units of vitamin D;
about 0.5 wt % of salt; and
about 0.7 wt % of olive oil and sunflower oil;
where the almond is a supareil almond and the almond size 23/25.

### Methods of Preparation

In some aspects, the disclosure provides a method for preparing a nut or seed product including a coating, where the coating includes a nutritional additive.

In some aspects, the disclosure provides a method for preparing a nut or seed product including a coating, where the coating includes a nutritional additive, the method including:
a roasting step; and
a seasoning step, where the seasoning step includes adding a coating including a nutritional additive to the nut or seed product.

In some aspects, the disclosure provides a method for preparing an almond including a coating, where the coating includes a nutritional additive.

In some aspects, the disclosure provides a method for preparing an almond including a coating, where the coating includes a nutritional additive, the method including:
a roasting step; and
a seasoning step, where the seasoning step includes adding a coating including a nutritional additive to the almond.

In some embodiments, the roasting step includes heating the almonds.

In some embodiments, the roasting step includes heating the almonds at a temperature of about 250° F. to about 400° F., about 275° F. to about 375° F., or about 300° F. to about 350° F. In some embodiments, the roasting step includes heating the almonds at a temperature of about 250° F. to about 400° F. In some embodiments, the roasting step includes heating the almonds at a temperature of about 275° F. to about 375° F. In some embodiments, the roasting step includes heating the almonds at a temperature of about 300° F. to about 350° F.

In some embodiments, the roasting step includes heating the almonds at a temperature of about 250° F. In some embodiments, the roasting step includes heating the almonds at a temperature of about 275° F. In some embodiments, the roasting step includes heating the almonds at a temperature of about 300° F. In some embodiments, the roasting step includes heating the almonds at a temperature of about 325° F. In some embodiments, the roasting step includes heating the almonds at a temperature of about 350° F. In some embodiments, the roasting step includes heating the almonds at a temperature of about 375° F. In some embodiments, the roasting step includes heating the almonds at a temperature of about 400° F.

In some embodiments, the roasting step includes heating the almonds for about 4 to about 20 minutes, about 6 to about 16 minutes, or about 8 to about 12 minutes. In some embodiments, the roasting step includes heating the almonds for about 4 to about 20 minutes. In some embodiments, the roasting step includes heating the almonds for about 6 to about 16 minutes. In some embodiments, the roasting step includes heating the almonds for about 8 to about 12 minutes.

In some embodiments, the roasting step includes heating the almonds for about 4 minutes. In some embodiments, the roasting step includes heating the almonds for about 6 minutes. In some embodiments, the roasting step includes heating the almonds for about 8 minutes. In some embodiments, the roasting step includes heating the almonds for about 10 minutes. In some embodiments, the roasting step includes heating the almonds for about 12 minutes. In some embodiments, the roasting step includes heating the almonds for about 14 minutes. In some embodiments, the roasting step includes heating the almonds for about 16 minutes. In some embodiments, the roasting step includes heating the almonds for about 18 minutes. In some embodiments, the roasting step includes heating the almonds for about 20 minutes.

In some embodiments, the method further includes a blanching step.

In some embodiments, the blanching step includes boiling the nut or seed product in water, draining the nut or seed product, and rinsing the nut or seed product with cold water.

In some embodiments, the blanching step includes boiling the almond in water, draining the almond, and rinsing the almond with cold water.

In some embodiments, the blanching step further includes removing the skin from the nut or seed to provide a skinless nut or seed product.

In some embodiments, the blanching step further includes removing the skin from the almond to provide a skinless almond.

In some embodiments, the method further includes a packaging step.

In some embodiments, the nutritional additive is added in oil form. In some embodiments, the nutritional additive is added in powdered form. In some embodiments, vitamin D is added in oil form. In some embodiments, vitamin D is added in powdered form. In some embodiments, a solution of vitamin D in olive oil is added.

In some embodiments, about 800 to about 1250 international units of a nutritional additive is added per ounce of nut or seed. In some embodiments, about 900 to about 1100 international units of a nutritional additive is added per ounce of nut or seed. In some embodiments, about 1000 international units of a nutritional additive is added per ounce of nut or seed.

In some embodiments, about 800 to about 1250 international units of vitamin D is added per ounce of nut or seed. In some embodiments, about 900 to about 1100 international units of vitamin D is added per ounce of nut or seed. In some embodiments, about 1000 international units of vitamin D is added per ounce of nut or seed. In some embodiments, the nut or seed is an almond.

In some embodiments, the seasoning step further includes adding a flavor additive to the nut or seed. In some embodiments, the seasoning step further includes adding salt to the nut or seed. In some embodiments, the seasoning step further includes adding sea salt to the nut or seed. In some embodiments, the nut or seed is an almond.

In some embodiments, about 0.1 wt % to about 15.0 wt % of a flavor additive is added. In some embodiments, about 8.0 wt % of a flavor additive is added. In some embodiments, about 0.1 wt % to about 1.0 wt % of a flavor additive is added. In some embodiments, about 0.5 wt % of a flavor additive is added. In some embodiments, about 0.1 wt % to about 1.0 wt % of salt is added. In some embodiments, about 0.5 wt % of salt is added. In some embodiments, about 0.1 wt % to about 1.0 wt % of sea salt is added. In some embodiments, about 0.5 wt % of sea salt is added.

In some embodiments, the seasoning step further includes adding an oil. In some embodiments, the seasoning step further includes adding a tumbling oil. In some embodiments, the seasoning step further includes adding grapeseed oil. In some embodiments, the seasoning step further includes adding olive oil. In some embodiments, the seasoning step further includes adding sunflower oil. In some embodiments, the seasoning step further includes adding olive oil and sunflower oil.

### EXAMPLES

### Example 1. Preparation of Coated Almond Products

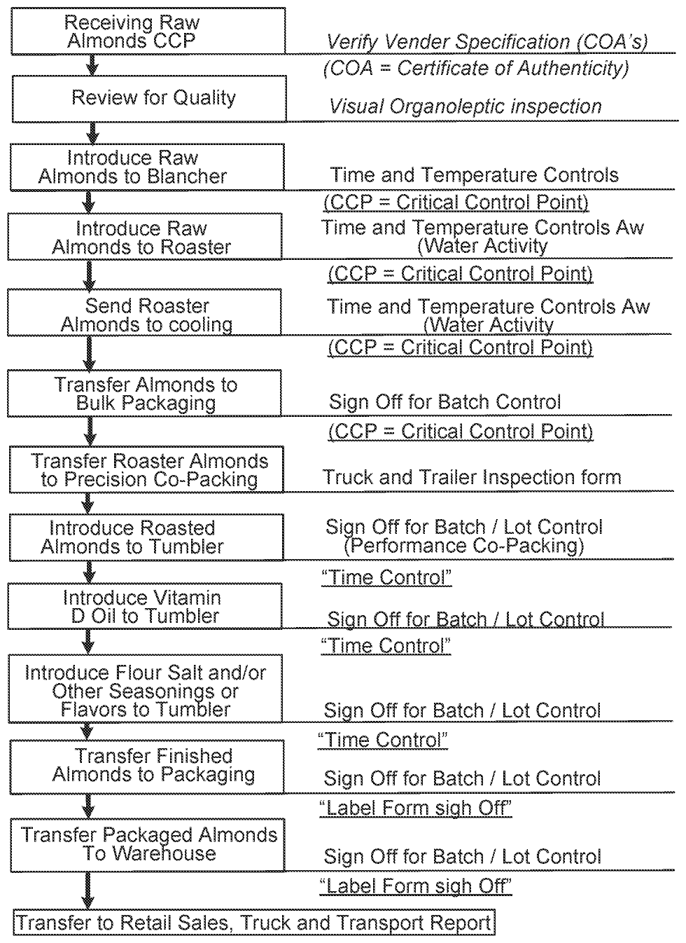

The coated almond products were prepared according to the procedure diagramed in FIG. 1. Large size nonpareil almonds (23 or 24 almonds per ounce) were hulled, shelled, blanched, dried, and lightly roasted by an almond processor. The almonds were then lightly oiled with vitamin D mixed oil at a ratio of about 1000 international units per one ounce of almonds. Approximately 0.5 wt % salt of salt was added. The almonds were then packaged.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

### Embodiments

Embodiment 1. A nut or seed product comprising a coating, wherein the coating comprises a nutritional additive and an oil.

Embodiment 2. The nut or seed product of Embodiment 1, wherein the nut or seed is selected from almond, peanut, pistachio, walnut, pecan, macadamia nut, cashew, pumpkin seed, and sunflower seed.

Embodiment 3. The nut or seed product of Embodiment 2, wherein the nut or seed is an almond.

Embodiment 4. The nut or seed product of any one of Embodiments 1-3, wherein the nutritional additive is selected from the group consisting of a vitamin, a mineral, an antioxidant agent, an anti-inflammatory agent, and combinations thereof.

Embodiment 5. The nut or seed product of any one of Embodiments 1-3, wherein the nutritional additive is a vitamin selected from the group consisting of vitamin A, vitamin C, vitamin D, vitamin E, vitamin K, vitamin B1 (thiamine), vitamin B2 (riboflavin), vitamin B3 (niacin), vitamin B6 (pyridoxine), vitamin B12 (cyanocobalamin), pantothenic acid (B5), biotin (B7), folate (folic acid or B9), and combinations thereof.

Embodiment 6. The nut or seed product of Embodiment 4 or 5, wherein the vitamin is vitamin D.

Embodiment 7. The nut or seed product of any one of Embodiments 1-3, wherein the nutritional additive is a mineral selected from the group consisting of calcium, phosphorus, potassium, sodium, iodine, iron, magnesium, selenium, sulfur, copper, fluoride, chloride, manganese, cobalt, and combinations thereof.

Embodiment 8. The nut or seed product of any one of Embodiments 1-3, wherein the nutritional additive is an antioxidant agent selected from the group consisting of vitamin C, vitamin E, beta-carotene, selenium, manganese, glutathione, coenzyme Q10 , lipoic acid, flavonoids, phenols, polyphenols, phytoestrogens, and combinations thereof.

Embodiment 9. The nut or seed product of any one of Embodiments 1-3, wherein the nutritional additive is an anti-inflammatory agent selected from the group consisting of omega-3 fatty acid, curcumin, S-adenosylmethionine, zinc, green tea, frankincense, capsaicin, cat's claw, and combinations thereof.

Embodiment 10. The nut or seed product of any one of Embodiments 1-9, comprising about 200 to about 1800 international units, about 500 to about 1500 international units, about 600 to about 1400 international units, about 700 to about 1300 international units, about 800 to about 1200 international units, about 800 to about 1250 international units, or about 900 to about 1100 international units of the nutritional additive per ounce of nut or seed.

Embodiment 11. The nut or seed product of any one of Embodiments 1-9, comprising about 400 international units of the nutritional additive per ounce of nut or seed.

Embodiment 12. The nut or seed product of any one of Embodiments 1-9, comprising about 1000 international units of the nutritional additive per ounce of nut or seed.

Embodiment 13. The nut or seed product of any one of Embodiments 1-12, wherein the nut or seed is skinless and/or shelled.

Embodiment 14. The nut or seed product of any one of Embodiments 1-13, wherein the nut or seed is blanched.

Embodiment 15. The nut or seed product of any one of Embodiments 1-14, wherein the nut or seed is roasted.

Embodiment 16. The nut or seed product of any one of Embodiments 1-15, wherein the nut or seed is an almond, and the almond is a sweet almond.

Embodiment 17. The nut or seed product of Embodiment 16, wherein the sweet almond is selected from the group consisting of a nonpareil almond, a supareil almond, a Sonora almond, a Winters almond, an Avalon almond, a Durango almond, an Aldrich almond, a Price almond, a Wood Colony almond, a Carmel almond, a Monterey almond, a Butte almond, a Padre almond, a Mission almond, and a Ruby almond.

Embodiment 18.

The nut or seed product of Embodiment 17, wherein the almond is a nonpareil almond.

Embodiment 19. The nut or seed product of Embodiment 17, wherein the almond is a supareil almond.

Embodiment 20. The nut or seed product of any one of Embodiments 3 and 16-19, wherein the almond size is 16/18, 18/20, 20/22, 22/24, 23/25, 25/27, 27/30, 30/32, 32/34 or 36/40.

Embodiment 21. The nut or seed product of Embodiment 20, wherein the almond size is 23/25.

Embodiment 22. The nut or seed product of any one of Embodiments 1-21, wherein the oil is selected from the group consisting of vegetable oil, olive oil, canola oil, grapeseed oil, grape seed oil extract, sunflower oil, safflower oil, and combinations thereof.

Embodiment 23. The nut or seed product of Embodiment 21, wherein the oil is selected from grapeseed oil, olive oil, sunflower oil, or combinations thereof.

Embodiment 24. The nut or seed product of any one of Embodiments 1-23, further comprising a flavor additive selected from salt, black pepper, garlic powder, Mexican spice, everything bagel seasoning, dark chocolate coconut, dark chocolate, honey cinnamon, cinnamon, 5 pepper blend, and combinations thereof.

Embodiment 25. The nut or seed product of any one of Embodiments 1-23, further comprising a flavor additive selected from salt, black pepper, garlic powder, and combinations thereof.

Embodiment 26. The nut or seed product of Embodiment 24 or 25, wherein the flavor additive is salt.

Embodiment 27. The nut or seed product of Embodiment 26, wherein the salt is sea salt.

Embodiment 28. The nut or seed product of any one of Embodiments 24-27, comprising about 0.1 wt % to about 15.0 wt %, about 2.0 wt % to about 12.0 wt %, about 4.0 wt % to about 10.0 wt %, about 6.0 wt % to about 9.0 wt %, or about 7.5 wt % to about 8.5 wt % of the flavor additive.

Embodiment 29. The nut or seed product of any one of Embodiments 24-27, comprising about 8.0 wt % of the flavor additive.

Embodiment 30. The nut or seed product of any one of Embodiments 24-27, comprising about 0.01 wt % to about 2.0 wt %, about 0.05 wt % to about 1.5 wt %, about 0.1 wt % to about 1.0 wt %, about 0.2 wt % to about 0.8 wt %, about 0.3 wt % to about 0.7 wt %, or about 0.4 wt % to about 0.6 wt % of the flavor additive.

Embodiment 31. The nut or seed product of any one of Embodiments 24-27, comprising about 0.5 wt % of the flavor additive.

Embodiment 32. A skinless almond comprising a coating, wherein the coating comprises about 300 to about 500 international units of a nutritional additive per ounce of almond, wherein the nutritional additive is vitamin D.

Embodiment 33. A skinless almond comprising a coating, wherein the coating comprises about 400 international units of a nutritional additive per ounce of almond, wherein the nutritional additive is vitamin D.

Embodiment 34. A skinless almond comprising a coating, wherein the coating comprises about 800 to about 1250 international units of a nutritional additive per ounce of almond, wherein the nutritional additive is vitamin D.

Embodiment 35. A skinless almond comprising a coating, wherein the coating comprises about 900 to about 1100 international units of a nutritional additive per ounce of almond, wherein the nutritional additive is vitamin D.

Embodiment 36. A skinless almond comprising a coating, wherein the coating comprises about 1000 international units of a nutritional additive per ounce of almond, wherein the nutritional additive is vitamin D.

Embodiment 37. A skinless, blanched, roasted almond comprising a coating, wherein the coating comprises:
about 800 to about 1250 international units of a nutritional additive per ounce of almond, wherein the nutritional additive is vitamin D;
about 0.2 wt % to about 1.2 wt % sunflower oil; and
about 0.1 wt % to about 1.0 wt % of a flavor additive, wherein the flavor additive is sea salt;
wherein the almond is size 23/25.

Embodiment 38. A skinless, blanched, roasted almond comprising a coating, wherein the coating comprises:
about 900 to about 1100 international units of a nutritional additive per ounce of almond, wherein the nutritional additive is vitamin D;
about 0.2 wt % to about 1.2 wt % sunflower oil; and
about 0.1 wt % to about 1.0 wt % of a flavor additive, wherein the flavor additive is sea salt;
wherein the almond is size 23/25.

Embodiment 39. A skinless, blanched, roasted almond comprising a coating, wherein the coating comprises:
about 300 to about 500 international units of a nutritional additive per ounce of almond, wherein the nutritional additive is vitamin D;
about 0.2 wt % to about 1.2 wt % sunflower oil; and
about 0.1 wt % to about 1.0 wt % of a flavor additive, wherein the flavor additive is sea salt;
wherein the almond is size 23/25.

Embodiment 40. A method of preparing the nut or seed product of any one of Embodiments 1-39, comprising:
a blanching step;
a roasting step; and
a seasoning step, comprising adding a coating comprising a nutritional additive to the nut or seed product.

Embodiment 41. The method of Embodiment 40, wherein the nut or seed product is almond.

Embodiment 42. The method of Embodiment 41, wherein the nutritional additive is added in oil form.

Embodiment 43. The method of Embodiment 41, wherein the nutritional additive is added in powdered form.

Embodiment 44. The method of any one of Embodiments 40-43, wherein the nutritional additive is vitamin D.

Embodiment 45. The method of any one of Embodiments 40-44, comprising adding about 800 to about 1250 international units of a nutritional additive per ounce of nut or seed product.

Embodiment 46. The method of any one of Embodiments 40-44, comprising adding about 900 to about 1100 international units of a nutritional additive per ounce of nut or seed product.

Embodiment 47. The method of any one of Embodiments 40-44, comprising adding about 300 to about 500 international units of a nutritional additive per ounce of nut or seed product.

Embodiment 48. The method of any one of Embodiments 40-47, wherein the seasoning step further comprises adding a flavor additive to the nut or seed product.

Embodiment 49. The method of any one of Embodiments 40-48, wherein the flavor additive is selected from salt, black pepper, garlic powder, Mexican spice, everything bagel seasoning, dark chocolate coconut, dark chocolate, honey cinnamon, cinnamon, 5 pepper blend, and combinations thereof.

Embodiment 50. The method of Embodiment 49, wherein the flavor additive is sea salt.

Embodiment 51. The method of Embodiment 48 or 50, comprising adding about 0.09 wt % to about 0.2 wt % of a flavor additive.

Embodiment 52. The method of any one of Embodiments 40-51, wherein the seasoning step further comprises adding a tumbling oil.

What is claimed is:

1. A nut or seed product comprising a coating, wherein the coating comprises a nutritional additive and an oil.

2. The nut or seed product of claim 1, wherein the nut or seed is an almond.

3. The nut or seed product of claim 2, wherein the nutritional additive is selected from the group consisting of a vitamin, a mineral, an antioxidant agent, an anti-inflammatory agent, and combinations thereof.

4. The nut or seed product of claim 3, wherein the vitamin is vitamin D.

5. The nut or seed product of claim 2, comprising about 200 to about 1800 international units, about 500 to about 1500 international units, about 600 to about 1400 international units, about 700 to about 1300 international units, about 800 to about 1200 international units, about 800 to about 1250 international units, or about 900 to about 1100 international units of the nutritional additive per ounce of nut or seed.

6. The nut or seed product of claim 5, comprising about 400 or about 1000 international units of the nutritional additive per ounce of nut or seed.

7. The nut or seed product of claim 2, wherein the nut or seed is skinless, shelled, blanched, and/or roasted.

8. The nut or seed product of claim 2, wherein the almond size is 23/25.

9. The nut or seed product of claim 2, further comprising an oil selected from grapeseed oil, olive oil, sunflower oil, or combinations thereof.

10. The nut or seed product of claim 2, further comprising a flavor additive selected from salt, black pepper, garlic powder, Mexican spice, everything bagel seasoning, dark chocolate coconut, dark chocolate, honey cinnamon, cinnamon, 5 pepper blend, and combinations thereof.

11. The nut or seed product of claim 10, wherein the flavor additive is salt, and the salt is sea salt.

12. The nut or seed product of claim 10, comprising about 0.1 wt % to about 15.0 wt %, about 2.0 wt % to about 12.0 wt %, about 4.0 wt % to about 10.0 wt %, about 6.0 wt % to about 9.0 wt %, or about 7.5 wt % to about 8.5 wt % of the flavor additive.

13. The nut or seed product of claim 12, comprising about 8.0 wt % or about 0.5 wt % of the flavor additive.

14. An almond comprising a coating, wherein:
the almond is skinless;
the coating comprises about 200 to about 1800 international units of a nutritional additive per ounce of almond; and
the nutritional additive is vitamin D.

15. The almond comprising a coating of claim 14, wherein:
the almond is blanched and roasted;
the almond is size 23/25; and
the coating comprises:
about 800 to about 1250 international units of a nutritional additive per ounce of almond, wherein the nutritional additive is vitamin D;
about 0.2 wt % to about 1.2 wt % oil; and
about 0.1 wt % to about 1.15 wt % of a flavor additive.

16. The almond comprising a coating of claim 14, wherein:
the almond is blanched and roasted;
the almond is size 23/25; and
the coating comprises:
about 300 to about 500 international units of a nutritional additive per ounce of almond, wherein the nutritional additive is vitamin D;
about 0.2 wt % to about 1.2 wt % sunflower oil; and
about 0.1 wt % to about 1.15 wt % of a flavor additive.

17. A method of preparing the nut or seed product of claim 1, comprising:
a blanching step;
a roasting step; and
a seasoning step, comprising adding a coating comprising a nutritional additive to the nut or seed product.

18. The method of claim 17, wherein the nut or seed product is almond and the nutritional additive is vitamin D.

19. The method of claim 17, comprising adding about 300 to about 500 or about 800 to about 1250 international units of a nutritional additive per ounce of nut or seed product.

20. The method of claim 19, further comprising adding a flavor additive, wherein the flavor additive is sea salt.
